(19) United States
(12) Patent Application Publication
(10) Pub. No.: US 2022/0378578 A1
(43) Pub. Date: Dec. 1, 2022

(54) DEVICES, SYSTEMS, AND METHODS FOR IMAGING AND IMPLANTING CARDIAC DEVICES

(71) Applicant: Boston Scientific Scimed, Inc., Maple Grove, MN (US)

(72) Inventors: Eric Michael Petersen, Maple Grove, MN (US); Jerry Wang, Edina, MN (US); James M. Anderson, Corcoran, MN (US); John M. Edgell, Plymouth, MN (US)

(21) Appl. No.: 17/824,291

(22) Filed: May 25, 2022

Related U.S. Application Data
(60) Provisional application No. 63/193,230, filed on May 26, 2021.

(51) Int. Cl. A61F 2/24; A61B 1/005; A61B 1/018; A61B 1/00; A61B 1/313

(52) U.S. Cl. CPC A61F 2/2466; A61B 1/0058; A61B 1/0057; A61B 1/018; A61B 1/00183; A61B 1/3137

(57) ABSTRACT

Devices, systems, and methods for delivering various implantable devices, such as heart valve implants, with the use of a steerable imaging element are described. An imaging element is positioned adjacent the implantable device and implant site, a relationship between the implantable device and the implant site is visualized and/or imaged, and the implantable device is anchored and/or adjusted with reference to the visualization and/or imaging thereof.

### CROSS-REFERENCE TO RELATED APPLICATIONS

This application claims the benefit of priority of U.S. Provisional Application No. 63/193,230, filed May 26, 2021, the entire disclosure of which is incorporated herein for all purposes.

### FIELD

The present disclosure relates generally to the field of implantable medical devices and imaging thereof. In particular, the present disclosure relates to medical devices, systems, and methods for cardiac treatment.

### BACKGROUND

Various diseases or defects affect proper functioning of the heart. Heart disease can cause the chambers of the heart and/or the valves therebetween to expand and weaken, dangerously affecting proper cardiac function such as blood flow. As a result of aging or disease, the chambers, such as the left ventricle, may dilate and the cardiac muscles, such as the papillary muscles, may be displaced. Heart valve incompetency is a serious problem. As a result of aging or disease, the left ventricle may dilate and the papillary muscles may be displaced, causing the mitral heart valve annulus to dilate excessively. In this state of dilation, the valve leaflets no longer effectively close, or coapt, during systolic contraction. Consequently, regurgitation (i.e., retrograde flow back across the valve that should be closed) of blood occurs during ventricular contraction, and cardia output may decrease as a result, with various associated risks of morbidity and mortality due to stroke, thrombosis, heart attack and extended recovery time.

Various devices are known for addressing heart valve incompetency, such as annuloplasty devices which reshape the valve annulus to bring together the valve leaflets to restore proper coaptation. In particular, various cardiovascular devices are available for minimally-invasive (such as transluminal, e.g., transcatheter) procedures, in contrast with invasive (such as open-surgery), for delivery and deployment of the device. For instance, various annuloplasty devices are configured for delivery and deployment transfemorally and transeptally to the mitral valve. Various components of the devices may be at different angles with respect to one another or with respect to the implant site (e.g., the heart valve annulus). Accordingly, various challenges exist with regard to providing visualization equipment with respect to such devices and to maintaining non-skewed, unblocked, and focused view of the device and/or the anatomy at the treatment site/implant site, such as by maintaining the viewing equipment substantially centered/concentric/coaxial or otherwise positioned as desired relative to the device and/or the anatomy to which the device is to be implanted. Another significant procedural challenge is the ability to image the device, the implant site (e.g., mitral valve annulus), and the specific target locations for anchor placement.

### SUMMARY

This summary of the disclosure is given to aid understanding, and one of skill in the art will understand that each of the various aspects and features of the disclosure may advantageously be used separately in some instances, or in combination with other aspects and features of the disclosure in other instances. No limitation as to the scope of the claimed subject matter is intended by either the inclusion or non-inclusion of elements, components, or the like in this summary.

In accordance with various principles of the present disclosure, a visualization/imaging system is provided with an implantable device to facilitate and enhance visualization and imaging during delivery and/or deployment of the implantable device.

In one aspect, an implantable device and a visualization/imaging system are delivered or are deliverable through a common lumen defined through an access sheath to an implant site. The distal end of a flexible elongate member of the visualization/imaging system is moved or is movable with respect to the longitudinal axis of the flexible elongate member to move an imaging element on the distal end of the visualization/imaging system relative to the implantable device and/or the implant site.

In some embodiments, the imaging element is moved or is movable to alter an angle of the imaging element relative to the implantable device and/or the implant site to affect visualization thereof.

In some embodiments, the implantable device is visualized and/or imaged with the visualization/imaging system, and a portion of the implantable device is anchored to the implant site based on visualizations and/or images from the visualization/imaging system.

In some embodiments, the implantable device is visualized and/or imaged with the visualization/imaging system, and a configuration of the implantable device is adjusted based on visualizations and/or images from the visualization/imaging system.

In some embodiments, the visualization/imaging system is delivered into an area circumscribed by the implantable device. The implantable device may be delivered with the use of a delivery/deployment system having a delivery lumen, the visualization/imaging system being delivered through the delivery lumen of the delivery/deployment system.

In some embodiments, the flexible elongate member includes at least one shape memory section such that bending of the flexible portion of the flexible elongate member is achieved without application of external force to the flexible elongate member.

In some embodiments, a steering mechanism is used or is usable to bend the flexible portion of the flexible elongate member.

In some embodiments, the imaging element is moved or is movable to view a distal side of the implantable device and/or the implant site.

In one aspect, an implantable device is delivered or is deliverable to an implant site with a visualization/imaging system adjacent the implantable device, the visualization/imaging system including a flexible portion on which at least one imaging element is positioned. The implantable device is visualized and/or imaged with the visualization/imaging system. The flexible portion of the flexible elongate member is bended or is bendable to alter the position of the at least one imaging element relative to the implantable device and/or the implant site to adjust the visualizing and/or imaging performed by the visualization/imaging system.

In some embodiments, a portion of the implantable device is anchored to the implant site based on visualizations and/or images from the visualization/imaging system.

In some embodiments, a configuration of the implantable device is adjusted based on visualizations and/or images from the visualization/imaging system.

In some embodiments, the visualization/imaging system is delivered or is deliverable into an area circumscribed by the implantable device.

In some embodiments, the flexible elongate member includes at least one shape memory section such that bending of the flexible portion of the flexible elongate member is achieved without application of external force to the flexible elongate member.

In some embodiments, a steering mechanism is used or is usable to bend the flexible portion of the flexible elongate member, such as to alter the angle of the at least one imaging element relative to the implantable device and/or the implant site.

In some embodiments, the imaging element is moved or is movable to view a distal side of the implantable device and/or the implant site.

In one aspect, a system for implanting an implantable device at an implant site includes an implantable device circumscribing an area, and a visualization/imaging system with at least one imaging element positionable within the circumscribed area within the implantable device and movable with respect to the implantable device to alter the angle of the imaging element relative to a portion of the implantable device to enhance visualization or imaging of the portion of the implantable device.

In some embodiments, the visualization/imaging system includes a flexible elongate member extending along longitudinal axis and having a flexible portion. The at least one imaging element is positioned adjacent the flexible portion of the flexible elongate member, and the flexible portion of the flexible elongate member is bended or is bendable to alter the angle of the imaging element relative to the longitudinal axis of the flexible elongate member and the implantable device.

In some embodiments, the visualization/imaging system includes a flexible elongate member extending along a longitudinal axis, and the at least one imaging element includes a first imaging element on a distal end of the flexible elongate member and a second imaging element positioned adjacent and proximal to the first imaging element. In some embodiments, the flexible elongate member is bended or is bendable between the first imaging element and the second imaging element to alter the angle between the first and second imaging elements to visualize and/or image different portions of the implantable device.

These and other features and advantages of the present disclosure, will be readily apparent from the following detailed description, the scope of the claimed invention being set out in the appended claims. While the following disclosure is presented in terms of aspects or embodiments, it should be appreciated that individual aspects can be claimed separately or in combination with aspects and features of that embodiment or any other embodiment.

### BRIEF DESCRIPTION OF THE DRAWINGS

Non-limiting embodiments of the present disclosure are described by way of example with reference to the accompanying drawings, which are schematic and not intended to be drawn to scale. The accompanying drawings are provided for purposes of illustration only, and the dimensions, positions, order, and relative sizes reflected in the figures in the drawings may vary. For example, devices may be enlarged so that detail is discernable, but is intended to be scaled down in relation to, e.g., fit within a working channel of a delivery catheter or endoscope. In the figures, identical or nearly identical or equivalent elements are typically represented by the same reference characters, and similar elements are typically designated with similar reference numbers differing in increments of 100, with redundant description omitted. For purposes of clarity and simplicity, not every element is labeled in every figure, nor is every element of each embodiment shown where illustration is not necessary to allow those of ordinary skill in the art to understand the disclosure.

The detailed description will be better understood in conjunction with the accompanying drawings, wherein like reference characters represent like elements, as follows:

FIG. 1 is a schematic view of a human heart with an example of a delivery/deployment system delivering an implantable device to a mitral valve annulus.

FIG. 2 is a partial perspective view of a distal portion of a delivery/deployment system implanting and adjusting an implantable device in accordance with aspects of the present disclosure.

FIG. 3A is a partial perspective view of a distal portion of a delivery/deployment system implanting and adjusting an implantable device with an embodiment of an imaging system in accordance with aspects of the present disclosure.

FIG. 3B is a perspective view of an imaging device as in FIG. 3A in another position relative to the implantable device.

FIG. 4 is a an elevational view of an example of an embodiment of an imaging device which may be used with a delivery/deployment system as in FIG. 2 or 3.

FIG. 5 is an elevational view of another example of an embodiment of an imaging device which may be used with a delivery/deployment system as in FIG. 2 or 3.

FIG. 6 is an elevational view of another example of an embodiment of an imaging device which may be used with a delivery/deployment system as in FIG. 2 or 3.

FIG. 7 is an elevational view of another example of an embodiment of an imaging device which may be used with a delivery/deployment system as in FIG. 2 or 3.

FIG. 8A is a perspective view of an imaging device with an embodiment of a steering mechanism in accordance with various principles of the present disclosure.

FIG. 8B is a perspective view of a steering mechanism as in FIG. 8A deflecting an imaging device as in FIG. 8A upward in accordance with various principles of the present disclosure.

FIG. 8C is a perspective view of a steering mechanism as in FIG. 8A deflecting an imaging device as in FIG. 8A downward in accordance with various principles of the present disclosure.

FIG. 9 is cross-sectional view of an example of a handle for controlling a steering mechanism in accordance with various principles of the present disclosure.

FIG. 10 is an exploded of an example of a handle for controlling a steering mechanism in accordance with various principles of the present disclosure.

FIG. 11 is a perspective view of another example of a handle for controlling a steering mechanism in accordance with various principles of the present disclosure.

FIG. 12 is a perspective view of another example of a handle for controlling a steering mechanism in accordance with various principles of the present disclosure.

### DETAILED DESCRIPTION

The following detailed description should be read with reference to the drawings, which depict illustrative embodiments. It is to be understood that the disclosure is not limited to the particular embodiments described, as such may vary. All apparatuses and systems and methods discussed herein are examples of apparatuses and/or systems and/or methods implemented in accordance with one or more principles of this disclosure. Each example of an embodiment is provided by way of explanation and is not the only way to implement these principles but are merely examples. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. In fact, it will be apparent to those skilled in the art that various modifications and variations can be made in the present disclosure without departing from the scope or spirit of the present subject matter. For instance, features illustrated or described as part of one embodiment can be used with another embodiment to yield a still further embodiment. Thus, it is intended that the present subject matter covers such modifications and variations as come within the scope of the appended claims and their equivalents.

It will be appreciated that the present disclosure is set forth in various levels of detail in this application. In certain instances, details that are not necessary for one of ordinary skill in the art to understand the disclosure, or that render other details difficult to perceive may have been omitted. The terminology used herein is for the purpose of describing particular embodiments only, and is not intended to be limiting beyond the scope of the appended claims. Unless defined otherwise, technical terms used herein are to be understood as commonly understood by one of ordinary skill in the art to which the disclosure belongs. All of the devices and/or methods disclosed and claimed herein can be made and executed without undue experimentation in light of the present disclosure.

As used herein, “proximal” refers to the direction or location closest to the user (medical professional or clinician or technician or operator or physician, etc., such terms being used interchangeably herein without intent to limit, and including automated controller systems or otherwise), etc., such as when using a device (e.g., introducing the device into a patient, or during implantation, positioning, or delivery), and “distal” refers to the direction or location furthest from the user, such as when using the device (e.g., introducing the device into a patient, or during implantation, positioning, or delivery). “Longitudinal” means extending along the longer or larger dimension of an element. “Central” means at least generally bisecting a center point and/or generally equidistant from a periphery or boundary, and a “central axis” means, with respect to an opening, a line that at least generally bisects a center point of the opening, extending longitudinally along the length of the opening when the opening comprises, for example, a tubular element, a strut, a channel, a cavity, or a bore.

In accordance with various principles of the present disclosure, devices, systems, and methods for deploying an implantable device within a body include use of a visualization and/or imaging system which is integrated with the system used to deliver and/or deploy the implantable device to the intended treatment site/implant site. As used herein, such terms as treatment site or implant site may be used interchangeably, without intent to limit, to refer to the location within the body to which an implantable device is delivered and to be implanted. It will be appreciated that terms such as deploy and implant (and conjugations and other grammatical forms thereof) may be used interchangeably herein with terms such as anchor, affix, fix, secure, couple, engage, hold, retain, etc., (and various conjugations and other grammatical forms thereof) without intent to limit.

More particularly, in accordance with various principles of the present disclosure, a visualization and/or imaging system may be navigated with, delivered with, coupled to, or otherwise associated with the system used to deliver and/or deploy the implantable device to be used therewith. Integration of a visualization and/or imaging system with an implantable device may provide various advantages. For instance, in contrast with the use of transesophageal echocardiography (“TEE”), a visualization and/or imaging system integrated with an implantable device allows imaging from the treatment site (e.g., within or adjacent the implantable device), thereby allowing clearer, more accurate imaging. Further, use of a visualization and/or imaging system which allows movement of the imaging element (e.g., the component of the visualization and/or imaging system receiving images, such as the distal end or tip or free end of a fiber optic, or a transducer of an ultrasound imaging device) relative to the implantable device and/or the implant site provides even further benefits over prior devices, systems, and methods. Principles of the present disclosure may be applied to improve and to enhance visualization and imaging of various portions and components of an implantable device during deployment and implantation, as well as various regions of the implant site, thereby facilitating and improving upon (such as with regard to speed, efficiency, accuracy, etc.) prior deployment and implanting devices, systems, and methods.

As may be appreciated by those of ordinary skill in the art, visualization systems and imaging systems may have overlapping or common features and functions, such as the ability to determine the position, location, shape, size, configuration, etc. of an object in the field of view of the visualization or imaging system. A visualization system may or may not also generate images, and an imaging system may generate images or data which are not direct visual images of what is in the field of view thereof. For instance, fiber optics may be used as a conductor of a signal for visualizing, and may not be a transducer or emitter generating further images or data. Imaging instruments, such as intracardiac echocardiography (“ICE”), may be used to generate images which may not necessarily be considered visual images (not visual images representing the appearance such as to the human eye). It will be appreciated that reference is made to either or both a visualization system or an imaging system without intent to limit, reference being made to visualization/imaging system for the sake of convenience and without intent to limit to either. The principles of the present disclosure are applicable to either system and are not limited to one or the other system. It will further be appreciated that such references are to be understood as including other similar systems known or heretofore known in the art useful for providing information with regard to the position, location, shape, size, geometry, configuration, features, etc. of an implantable device in vivo to facilitate deployment of the device to implant the device at the treatment site.

In accordance with various principles of the present disclosure, an implantable device is delivered and deployed in conjunction with and the assistance of a visualization and/or imaging system, such as via a common delivery component. For instance, a common access sheath or catheter may be configured to deliver both the implantable device as well as the visualization and/or imaging system. Reference is made herein to a delivery/deployment system generically to reference a system used to deliver and/or deploy an implantable device. In some embodiments, a delivery sheath or catheter is used in conjunction with the delivery/deployment system, such as to navigate and/or deliver and/or control the delivery/deployment system. It will be appreciated that the terms sheath, catheter, tubular element, etc., may be used interchangeably herein without intent to limit. The devices, systems, and methods described herein allow bending of sheaths used therewith to occur without kinking or wrinkling to allow unimpeded transport and delivery of the implantable device through tortuous pathways through the body. Various configurations of a flexible sheath or portion or region thereof are within the scope and spirit of the present disclosure, such as described in (but not limited to) U.S. Pat. No. 10,335,275, issued on Jul. 2, 2019.

In some embodiments, the implantable device circumscribes an area (not necessarily circular), and the visualization and/or imaging system is positioned within such circumscribed area. In some embodiments, the visualization and/or imaging system is delivered with the implantable device, such as within such circumscribed area. In some embodiments, the visualization and/or imaging system is extended into such circumscribed area upon (e.g., after) delivery of the implantable device to the treatment site.

In accordance with various further principles of the present disclosure, the position and/or orientation of the visualization and/or imaging system is adjustable relative to the implantable device and/or the treatment site to facilitate proper placement and anchoring of the implantable device. More particularly, placement and adjustability of a visualization and/or imaging system relative to an implantable device and/or the implant site in accordance with various principles of the present disclosure may be used to verify the positioning of an anchoring element with respect to the implant site, the condition of the tissue to which the implantable device is to be secured, the depth of insertion of an anchoring element of the device, etc., and thus facilitates proper placement and anchoring of the implantable device in manners not as readily achievable previously. For instance, in some embodiments the imaging element( ) is movable with respect to the implantable device. In some embodiments, the imaging element is provided at or along or adjacent or on a flexible elongate member (such as an imaging catheter), at least a portion of the flexible elongate member being movable to move the imaging element with respect to the implantable device and/or implant site to provide information to the medical professional useful or necessary for implanting the device. It will be appreciated that terms such as at, along, adjacent, on, etc. are used interchangeably herein to cover specifically “at” as well as immediately adjacent as well as adjacent and spaced apart from and other such terms or spatial relationships. In some embodiments, the imaging element is moved to adjust the distance or spacing between the imaging element and the implantable device and/or the implant site. In some embodiments, the imaging element is moved to adjust the angular position thereof relative to the implantable device and/or the implant site, such as by rotating the imaging element. Additionally or alternatively, in some embodiments, the imaging element is moved to adjust the angular position thereof relative to the implantable device and/or the implant site, such as by adjusting the angle of the imaging element relative to the longitudinal axis of the flexible elongate member delivering or moving the imaging element. For instance, it may be desirable to position an imaging element relative to the implantable device and/or implant site to optimize visualization and/or imaging of the implantable device and/or implant site. In some embodiments, it may be desirable to align an imaging element with (e.g., to be parallel to) a portion of the implant (e.g., a strut or anchor) to enhance visualization and/or imaging. Any or all such movements of the imaging element generally improve the imaging quality and generally increase the information usable to deploy the implantable device effectively and successfully and efficiently (such as to reduce procedure time with concurrent benefits to the patient). For the sake of convenience, and without intent to limit, reference is made to the distal end of the flexible elongate member as the portion on which the imaging element is positioned.

In some embodiments, the imaging element is provided on a deflectable or steerable element, such as a flexible elongate member. At least a distal end of the deflectable flexible elongate member may be formed of a flexible material allowing flexing or bending of the flexible elongate member to deflect the imaging element carried by the flexible elongate member to a desired position for imaging an implantable device and/or implant site. It will be appreciated that terms such as deflect, flex, bend, curve, etc., and conjugations and other grammatical forms thereof, may be used interchangeably herein without intent to limit. One or more bends may be made or formed in the flexible elongate member, references to “a” bend being understood as not limited to a single bend. As such, a multidirectional, compound curve is contemplated.

In some embodiments, the flexible elongate member is a deflectable flexible elongate member formed of a shape memory or heat formable material (reference being made to simply shape memory for the sake of convenience and without intent to limit) which is delivered in a substantially straight or extended configuration (such as to facilitate transluminal delivery) and which bends to a deflected configuration once positioned adjacent the implantable device and/or the implant site. More particularly, an end region of the deflectable flexible elongate member carrying an imaging element may be pre-shaped or preformed from a shape memory material that bends from a generally straight delivery configuration to a deflected configuration, such as transverse to the longitudinal axis of the deflectable elongate member (i.e., the longitudinal axis of the proximally extending portion of the deflectable elongate member). Such bending modifies the position of the imaging element with respect to the longitudinal axis of the deflectable flexible elongate member and with respect to the implantable device and/or implant site to alter the visualization or image generated by the visualization/imaging system. The use of a shape memory material allows such bending to occur without the application of an external force. In other words, the bending occurs generally on its own (automatically) such as a result of internal forces in the material. In some embodiments, a compliant material may be used in conjunction with the shape memory material to facilitate bending of the deflectable flexible elongate member. Generally, the shape memory material overpowers the compliant material (e.g., the compliant material has a lower durometer or is less stiff than the shape memory material) to flex the deflectable flexible elongate member into the desired configuration. Additionally or alternatively, the deflectable flexible elongate member may include an elastomeric material under tension, optionally in combination with a compliant material. It will be appreciated that other suitable materials, such as electroactive polymers or metals to actuate bending or deflection, as known or heretofore known in the art may be used within the scope of the present disclosure. The use of a deflectable flexible elongate member may allow for improved positioning and angling of the imaging elements relative to the implantable device and/or the implant site.

In some embodiments, a portion of the flexible elongate member (whether or not deflectable on its own accord as described above) is moved by an external force. For example, a steering mechanism may be used to steer the flexible elongate member to position the imaging element with respect to the implantable device and/or the implant site to achieve the optimal views to facilitate deployment of the implantable device. It will be appreciated that terms such as steer, maneuver, navigate, deflect, manipulate, control, etc., and conjugations and other grammatical forms thereof, may be used interchangeably herein without intent to limit. The steering mechanism may be used to bend or simply to move (without bending, such as translate laterally) or to rotate (e.g., about the longitudinal axis of the flexible elongate member) the flexible elongate member to a desired position with respect to the implantable device and/or the implant site to facilitate deployment. It will be appreciated that the flexible elongate member steered by the steering mechanism need not include or be formed of a shape memory material. More particularly, a steering mechanism may steer a flexible elongate member which is deflected upon actuation of the steering mechanism but which does not deflect in a selected direction on its own. Alternatively, a steering mechanism may steer a flexible elongate member which deflects on its own, and which further deflects in a desired direction upon actuation of the steering mechanism.

Various configurations of steering mechanisms are contemplated to facilitate steering of the flexible elongate member to alter the position and/or orientation of the imaging element preferably from a location proximal to the implantation site, such as outside the patient. For instance, one or more pull wires or steering wires (such terms being interchangeable, the term “steering” wire being used herein for the sake of convenience and without intent to limit) may be coupled to the flexible elongate member and extend proximally to a steering handle which may be controllable outside the patient's body (in which the implantable device is being implanted). In some embodiments, a pull ring couples the one or more wires to the flexible elongate member. In some embodiments, the steering handle is rotatable or slidable to move the steering wires to effect movement of the flexible elongate member to adjust the position and/or orientation of the imaging element.

It will be appreciated that a steerable flexible elongate member formed in accordance with various principles of the present disclosure need not be deflectable in the sense that an element or portion or region thereof is formed of a memory material which deflects such element or portion or region without external forces applied thereto (i.e., the element or portion or region as a result of the properties, such as internal forces, of the material). An element or portion or region of a visualization/imaging system formed in accordance with various principles of the present disclosure may be flexible without necessarily moving to a particular configuration on its own. A steering mechanism may be used to adjust the position of such element or portion or region.

In accordance with various principles of the present disclosure, more than one imaging element may be used to visualize/image the implantable device and implant site to facilitate deployment. In some embodiments, the more than one imaging elements are positioned at different angular positions with respect to a flexible elongate member carrying the imaging elements. In some embodiments, the more than one imaging elements are positioned at different positions along the longitudinal axis and/or about the circumference of a flexible elongate member carrying the imaging elements. In some embodiments, the position and/or orientation of at least one imaging element is adjustable relative to the implantable device and/or the implant site. In some embodiments, the position and/or orientation of at least one imaging element is adjustable relative to at least another imaging element. In some embodiments, the flexible elongate member carrying the one or more imaging elements is a deflectable elongate member. Such deflectable elongate member may be capable of being deflected to modify the position of at least one imaging element relative to the implantable device and/or the implant site and/or another imaging element. In some embodiments, the deflectable member is formed of a shape memory material which deflects to adjust the position and/or orientation at least one of the imaging elements. In some embodiments, the deflectable elongate member is steerable and optionally includes a shape memory material. In some embodiments, the elongate member is simply flexible without the use of a shape memory material.

In some embodiments, the distal end of the flexible elongate member includes a first distal end carrying a first imaging element and a second distal end carrying a second imaging element. For instance, in some embodiments, the flexible elongate member includes a primary flexible elongate member and a secondary flexible elongate member, such as a dual monorail flexible elongate member. The primary flexible elongate member may be a “mother” flexible elongate member with a lumen through which the secondary flexible elongate member or “child” flexible elongate member is extendable. Either or both flexible elongate members are rotatable to adjust the position and/or orientation of the associated imaging elements with respect to the implantable device and/or the implant site. Either or both flexible elongate members are deflectable and/or steerable. For instance, the primary flexible elongate member may be deflectable so that it deflects upon reaching the desired site. The primary flexible elongate member may additionally or alternatively be steerable. The secondary flexible elongate member generally is longitudinally translatable relative to the primary flexible elongate member and may be deflectable and/or steerable. In some embodiments, the flexible elongate member is bifurcated into a first distal end carrying a first imaging element and a second distal end carrying a second imaging element. Either or both distal ends may be deflectable. In some embodiments, the first distal end extends distally of the second distal end and is deflectable, and the second distal end may be compliant and steerable, but may not deflect upon deployment.

Various principles of the present disclosure may be advantageously applied in delivering and deploying (e.g., positioning and placing) an implantable device or component thereof (such as an anchor). Various principles of the present disclosure may further advantageously be applied to adjust the configuration of the implantable device before or during or after implantation, such as to reshape and/or to replace a heart valve annulus to which the implantable device has been anchored. For instance, various principles of the present disclosure are particularly advantageously applied in delivering and deploying an implantable device (such as an annuloplasty device, with or without a valve replacement component) with multiple anchor points (points at which the device is anchored or otherwise secured to tissue). Various principles of the present disclosure are also particularly advantageously applied in delivering and deploying an implantable device which is adjusted or manipulated once secured to tissue, such as to modify the shape, position, configuration, etc. of the tissue. However, it will be appreciated that principles of the present disclosure are applicable to other implantable devices, systems, and methods as well.

It will further be appreciated that various devices, systems, and associated methods in accordance with various principles of the present disclosure are disclosed in co-pending U.S. patent application Ser. No. ______ filed concurrently herewith [ATTORNEY DOCKET 2001.2691101], which application is hereby incorporated by reference herein in its entirety for all purposes.

Various embodiments of a visualization and/or imaging system which may be used in accordance with various principles of the present disclosure to visualize and/or image an implantable device and implant site to deploy the implantable device will now be described with reference to various examples of embodiments illustrated in the accompanying drawings. Reference in this specification to “one embodiment,” “an embodiment,” “some embodiments”, “other embodiments”, etc. indicates that one or more particular features, structures, and/or characteristics in accordance with principles of the present disclosure may be included in connection with the embodiment. However, such references do not necessarily mean that all embodiments include the particular features, structures, and/or characteristics, or that an embodiment includes all features, structures, and/or characteristics. Some embodiments may include one or more such features, structures, and/or characteristics, in various combinations thereof. Moreover, references to “one embodiment,” “an embodiment,” “some embodiments”, “other embodiments”, etc. in various places in the specification are not necessarily all referring to the same embodiment, nor are separate or alternative embodiments necessarily mutually exclusive of other embodiments. When particular features, structures, and/or characteristics are described in connection with one embodiment, it should be understood that such features, structures, and/or characteristics may also be used in connection with other embodiments whether or not explicitly described, unless clearly stated to the contrary. It should further be understood that such features, structures, and/or characteristics may be used or present singly or in various combinations with one another to create alternative embodiments which are considered part of the present disclosure, as it would be too cumbersome to describe all of the numerous possible combinations and subcombinations of features, structures, and/or characteristics. Moreover, various features, structures, and/or characteristics are described which may be exhibited by some embodiments and not by others. Similarly, various features, structures, and/or characteristics or requirements are described which may be features, structures, and/or characteristics or requirements for some embodiments but may not be features, structures, and/or characteristics or requirements for other embodiments. Therefore, the present invention is not limited to only the embodiments specifically described herein.

Turning now to the drawings, it will be appreciated that in common features are identified by common reference elements and, for the sake of brevity and convenience, and without intent to limit, the descriptions of the common features are generally not repeated. For purposes of clarity, not all components having the same reference number are numbered. Moreover, a group of similar elements may be indicated by a number and letter, and reference may be made generally to one or such elements or such elements as a group by the number alone (without including the letters associated with each similar element). It will be appreciated that, in the following description, elements or components similar among the various illustrated embodiments with reference numbers greater than 1000 are generally designated with the same reference numbers increased by a multiple of 1000 and redundant description is omitted. Moreover, certain features in one embodiment may be used across different embodiments and are not necessarily individually labeled when appearing in different embodiments.

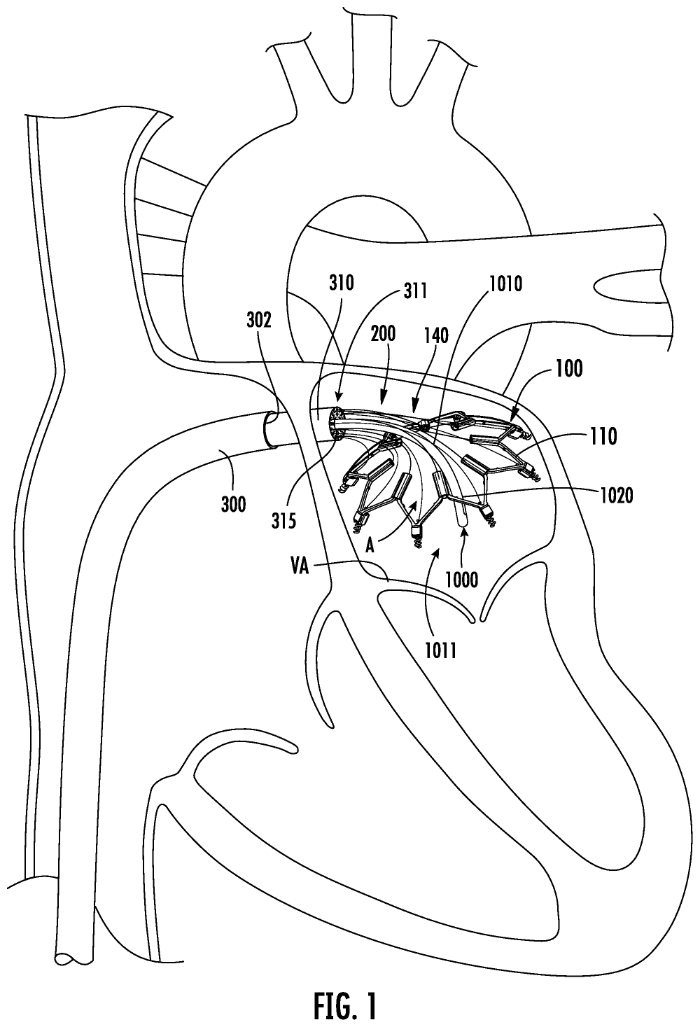

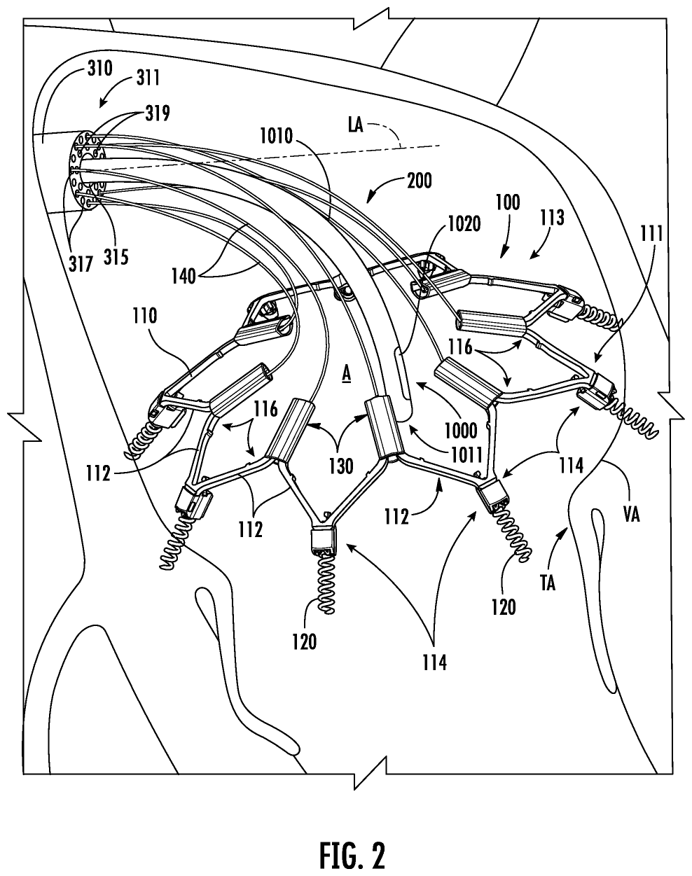

An example of an embodiment of an implantable device 100 being delivered by a delivery/deployment system 200 to a heart valve annulus VA (e.g., a mitral valve) in accordance with various principles of the present disclosure is illustrated in FIG. 1. The implantable device 100 is illustrated being positioned at the heart valve annulus VA by the delivery/deployment system 200 in FIG. 2 for deployment, such as via anchoring to the heart valve annulus VA. A visualization/imaging system 1000 is illustrated as being delivered with the implantable device 100 in accordance with various principles of the present disclosure. In the illustrated embodiment, the delivery/deployment system 200 and the visualization/imaging system 1000 are delivered via a common access catheter 300, and may be delivered through a common access lumen 302 of the access catheter 300. A delivery catheter 310 may be provided to facilitate delivery of the delivery/deployment system 200 and the visualization/imaging system 1000, with the implantable device 100 generally remaining outside and distal to the distal end 311 of the delivery catheter 310, as described in further detail below. The distal end 311 of the delivery catheter 310 is maneuvered into position above the heart valve annulus VA to position the implantable device 100 to be implanted to resize the heart valve annulus VA.

It is understood that a variety of different implantable devices may be delivered with a delivery system and methods described herein, only one example of which is illustrated and described herein. The embodiment of an implantable device 100 illustrated herein includes a frame 110 having a generally tubular shape circumscribing an area A, with the visualization/imaging system 1000 positioned within the circumscribed area A. The term “tubular” is to be understood herein to include circular as well as other rounded or otherwise closed shapes. The frame 110 may be configured to change shape, size, dimension, and/or configuration. For example, the frame 110 may assume various shapes, sizes, dimensions, configurations, etc. during different phases of deployment such as during pre-delivery, delivery, tissue engagement, anchoring, cinching, etc. The frame 110 may be formed from one or more struts 112 that may form all or part of the frame 110. The struts 112 may include elongated structural members formed of a metal alloy, a shape memory material, such as an alloy of nickel titanium or other metals, metal alloys, plastics, polymers, composites, other suitable materials, or combinations thereof. In one embodiment, the struts 112 may be formed from the same, monolithic piece of material (e.g., tube stock). Thus, reference to struts 112 may refer to different portions of the same, coextensive component. Alternatively, reference to struts 112 may refer to components that are formed separately and attached together (optionally permanently, such as by welding or other methods). In some embodiments, the struts 112 may be separate components that are detachably coupled to form distal apices 114 (which may alternately be referenced as distal/lower crowns 114) and proximal apices 116 (which may alternately be referenced as proximal/upper crowns 116). Alternatively, if formed from a monolithic piece of material, the material may be cut or otherwise formed to define distal apices 114 and proximal apices 116. The frame 110 carries a plurality of anchors 120 coupled to the distal apices 114 along a lower or distal portion 111 of the frame 110 and extending distally therefrom. Sliders 130 may extend over the proximal apices 116 along the upper or proximal portion 113 of the frame 110. The sliders 130 are slidable over the proximal apices 116 to adjust the relative position of the struts 112 forming the apices 116 to adjust the size, shape, or configuration of the frame 110. It will be appreciated that only some of the distal apices 114, proximal apices 116, struts 112, anchors 120, and sliders 130 are labelled for the sake of simplicity and clarity. The sliders may be referenced or otherwise known as collars or sleeves or cinch sleeves or nuts, and such terms may be used interchangeably herein without intent to limit, reference being made generally to sliders for the sake of convenience.

To adjust the size, shape, or configuration of the frame 110, and/or to anchor and position the implantable device 100, one or more driver shafts 140 may extend through the delivery catheter 310 to be extended from the distal end 311 of the delivery catheter 310 and coupled with the implantable device 100. The term driver shaft may be used interchangeably herein with terms such as driver, driver tube, actuator, etc., without intent to limit. The driver shafts 140 may include driver shafts 140 coupled to the anchors 120 and driver shafts 140 coupled to the sliders 130 in any desired manner known or heretofore known to permit movement (e.g., rotation or translation) of a driver shaft 140 to move a component (e.g., anchor 120 or slider 130) coupled thereto. Movement (e.g., rotation or translation) of driver shafts 140 engaged with the anchors 120 causes the anchors to move (e.g., rotate or translate) relative to the implant site, such as to engage and to penetrate into the tissue at the implant site to cause the frame 110 to be secured to the implant site. The anchors 120 may be advanced singly, one at a time (sequentially or in any desired order), or one or more anchors 120 may be advanced at the same time. Movement (e.g., translation or rotation) of driver shafts 140 engaged with the sliders 130 causes movement (e.g., translation or rotation) of the sliders 130 relative to the apices 116 to which the sliders 130 are coupled to move the orientations of the struts 112 to adjust the size, shape, and configuration of the frame 110. For the sake of simplicity, only the driver shafts 140 coupled to the sliders 130 are illustrated (to avoid the complexity of illustrating driver shafts 140 coupled to the anchors 120). The driver shafts 140 may extend through a delivery lumen 315 defined within the delivery catheter 310. In some embodiments, the delivery catheter 310 is formed with a multi-lumen extrusion 312 defining multiple longitudinally-extending lumens, such as device manipulation lumens 317 (through which the driver shafts 140 for adjusting the anchors 120 and/or the sliders 130 may extend) and/or steering control lumens 319 (through which steering wires described in further detail below may extend).

In the example of an embodiment illustrated in FIG. 1 and FIG. 2, the visualization/imaging system 1000 extends through the delivery lumen 315 defined through the delivery catheter 310, and the driver shafts 140 may extend through the device manipulation lumens 317 (such as to allow sufficient room for the visualization/imaging system 1000 to extend through the delivery lumen 315 without interference of the driver shafts 140). The visualization/imaging system 1000 may be extended from the distal end 311 of the delivery catheter 310 to within the circumscribed space A within the frame 110 during delivery, deployment, alignment, and/or positioning of the frame 110 above and proximate to the target heart valve annulus VA. In embodiments in which the driver shafts 140 extend through device manipulation lumens 317 of a multi-lumen extrusion, the visualization/imaging system 1000 may be extended through the delivery lumen 315 substantially unimpeded by the delivery/deployment system 200 into position with respect to the frame 110. The visualization/imaging system 1000 may be positioned with respect to the frame 110 (such as within the circumscribed area A defined within the frame 110) to view each region and/or apex of the implantable device 100. For instance, the visualization/imaging system 1000 may be positioned to view each anchor 120 with respect to the implant site, and/or placement of the anchors 120 and/or advancement of the anchors 120 (such as to confirm the desired extent of advancement). Further, the visualization/imaging system 1000 may be used to view various other components or features of the implantable device 100, such as the collars 130, for instance to view the extent to which each collar 130 is advanced down and over a proximal apex 116 of the frame 110, to more precisely adjust the size, shape, or configuration of the frame 110. It will be appreciated that terms such as view, visualize, image, etc. (and conjugations and other grammatical forms thereof) may be used interchangeably herein without intent to limit. The visualization/imaging system 1000 may also provide significant benefit to an embodiment in which a singular cinching mechanism or driver shaft 140 needs to be landed on each proximal apex 116 of the frame 110 to adjust the sizing of the frame 110.

The visualization/imaging system 1000 may be one or more fiber optics, an ultrasound catheter, an ICE catheter, or any other suitable device capable of transmitting an image to the operator of the delivery/deployment system 200 to facilitate delivery and/or deployment of the implantable device 100. Fiber optics may simply transmit visual signals or may replace wires carrying ultrasound signals to an imaging device for viewing by a medical professional. The visualization/imaging system 1000 may include a flexible elongate member 1010 and one or more imaging elements 1020 provided on or along a distal end 1011 of the flexible elongate member 1010. The imaging elements 1020 may be one or more linear imaging elements 1020 or one or more phased arrays of imaging elements 1020. Generally, a phased array of imaging elements 1020 is smaller and may be preferable for certain applications/environments. The one or more imaging elements 1020 may be arranged longitudinally along the longitudinal axis LA of the flexible elongate member 1010, and may include longitudinally spaced apart imaging elements 1020. Additionally or alternatively, the imaging elements 1020 may be arranged circumferentially about the perimeter of the flexible elongate member 1010, and may include circumferentially spaced apart imaging elements 1020. In some embodiments, rotation of the visualization/imaging system 1000 around the inside of the valve annulus, allows viewing of the relative position of the frame 110, and the implant site, such as the heart valve annulus, and/or any of the valve leaflets, for accurate positioning of the device and anchors 120 thereof relative to (e.g., around and above) the valve annulus VA. An indexing feature (not shown) on the visualization/imaging system 1000 may be provided such that actuation of the indexing feature causes the visualization/imaging system 1000 to move (e.g., rotate), optionally automatically, to the next position (such as from one anchor position to another anchor position).

The visualization/imaging system 1000 may thus be configured to generate/transmit a radial image, such as to position the one or more anchors 120 for insertion into a heart valve annulus VA, and/or a circumferential image, such as to position the frame 110 in a plane above the heart valve annulus VA and its leaflets. It will be appreciated that the devices and features shown and described herein may be used to deliver various other implantable devices, such as other resizing devices or heart valve replacement valves.

In some embodiments, software or electronic controls can be effective to cycle through the radial cross sectional images around the valve annulus perimeter, relieving the need to physically move (e.g., via rotation, translation, or deflection) the visualization/imaging system 1000. A larger circumferential imaging element array could also be placed distal of the annulus to not interfere with space limitations of the delivery catheter 310, further decreasing the profile of the delivery catheter 310. In another embodiment, the visualization/imaging system 1000 may generate a three dimensional image of the heart valve annulus VA and/or of the frame 110. The medical professional may then more readily see the relative alignment of the valve annulus, valve leaflets, and the implant device 100 (such as anchor points, e.g., anchor 120, thereof).

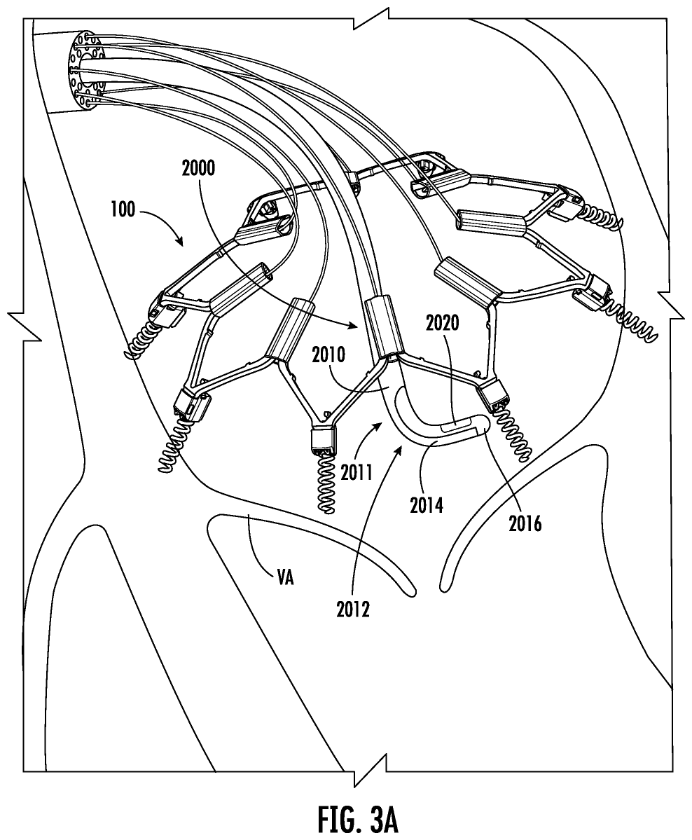

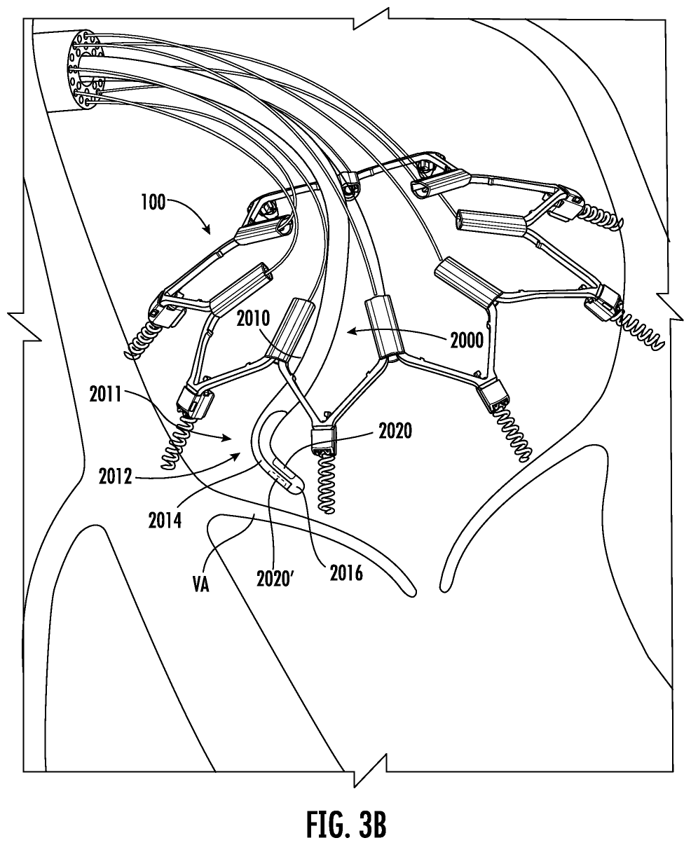

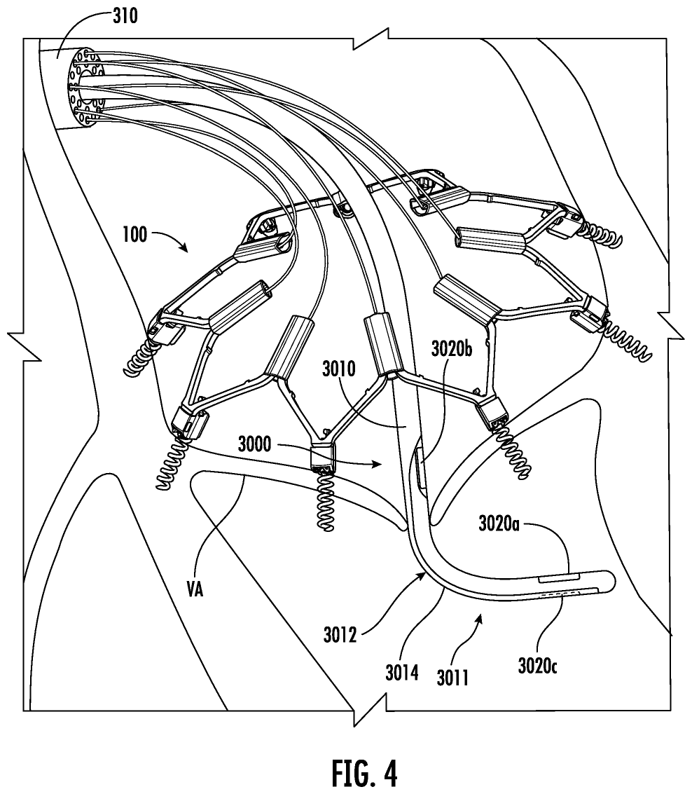

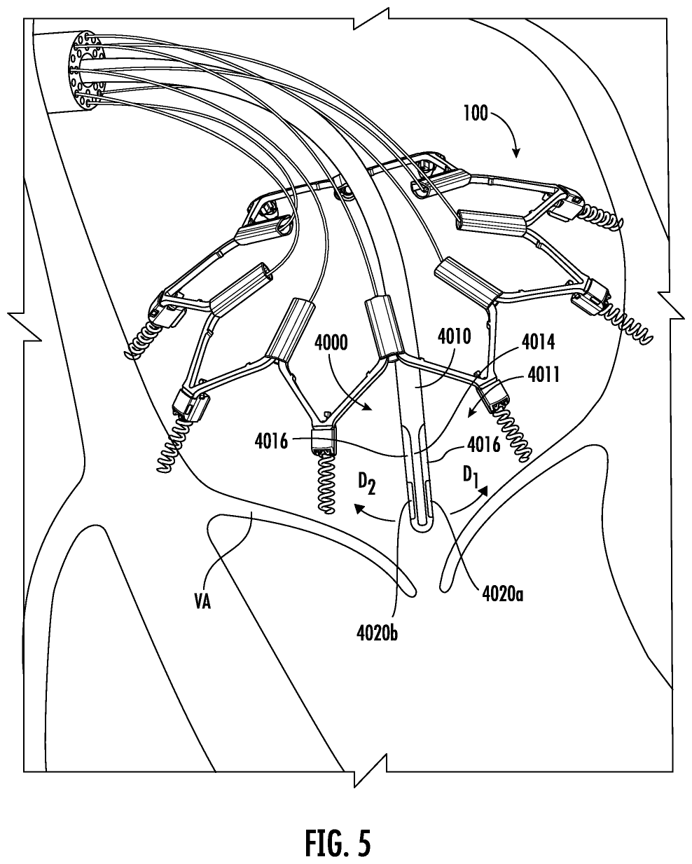

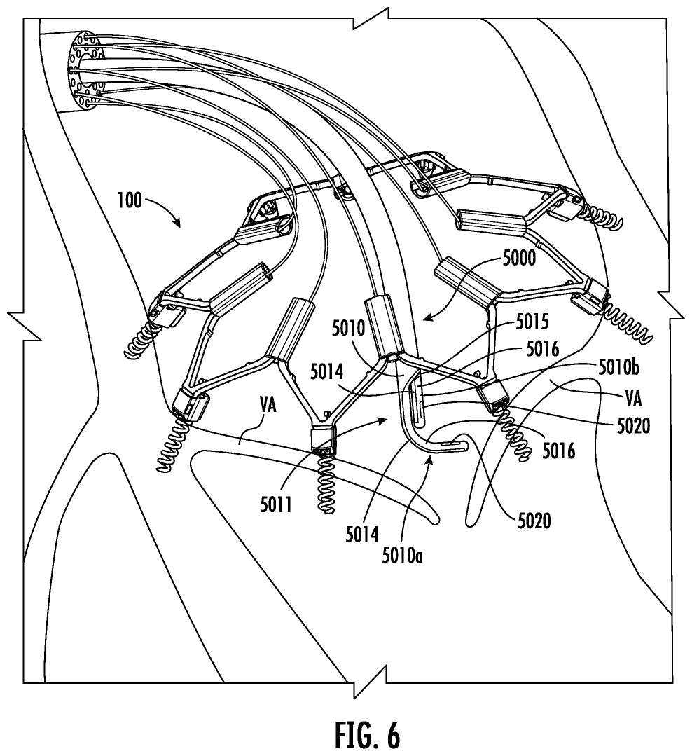

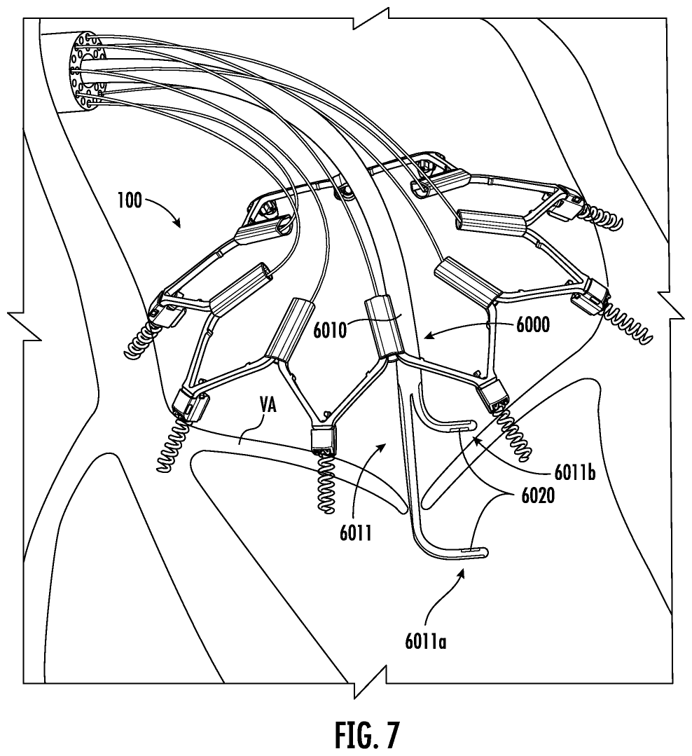

In accordance with various principles of the present disclosure, the flexible elongate member 1010 of a visualization/imaging system 1000 formed in accordance with various principles of the present disclosure may be sufficiently flexible to allow bending of the distal end 1011 thereof to adjust the position of the one or more imaging elements 1020 carried by the flexible elongate member 1010 relative to the implantable device 100 and/or the implant site. The resulting increased visualization may be used to verify the positioning of an anchor with respect to the heart valve annulus VA, the condition of the tissue of the heart valve V, the depth of insertion of an anchor, etc. In some embodiments, at least a portion of the distal end 1011 of the flexible elongate member 1010 is formed of a shape memory material causing the distal end 1011 to deflect to a preset configuration upon exiting the delivery lumen 315 of the delivery catheter 310. Such movement of the distal end 1011 of the flexible elongate member 1010 without external forces (i.e., movement caused by the internal forces of the material of the element itself) may be considered to form a self-deflecting, or, more simply, a deflectable visualization/imaging system 1000. Additionally or alternatively, in some embodiments, the flexible elongate member 1010 is steerable by a steering mechanism (e.g., a mechanism separately formed from the flexible elongate member 1010 to control or otherwise affect movement thereof). A steerable flexible elongate member 1010 optionally includes a shape memory material as well (to be deflectable), though need not include such material. A deflectable flexible elongate member 1010 formed at least in part from a shape memory material may include a compliant material positioned to facilitate flexing of the flexible elongate member 1010, but without itself necessarily moving or returning the flexible elongate member 1010 to a pre-selected/predetermined configuration. Various examples of embodiments and configurations of deflectable and/or steerable visualization/imaging systems will now be described with reference to FIG. 3A, FIG. 3B, FIG. 4, FIG. 5, FIG. 6, and FIG. 7.

In the example of an embodiment illustrated in FIG. 3A, a visualization/imaging system 2000 includes a deflectable flexible elongate member 2010 with a deflectable distal end 2011 carrying at least one imaging element 2020. The deflectable distal end 2011 includes a memory material section 2014 pre-shaped or pre-formed into a deflected configuration, such as to include a bend 2012. The memory material section 2014 may be formed of any known material with memory, exhibiting the desired properties and characteristics for biocompatibility; flexibility (for extending through tortuous pathways in the body); and deflectability into a predetermined desired shape (e.g., with a bent distal end relative to the remaining proximally extending portion of the flexible elongate member) generally without application of external forces thereto for facilitating viewing and/or imaging. A typical suitable material is a shape memory material such as nitinol or another metal or metal alloy or polymer, such as with similar properties. The memory material section 2014 may be extended into a substantially straight configuration (such as shown in FIG. 1 or FIG. 2) when within or at least partially within the delivery lumen 315 of the delivery catheter 310. Once extended out from (and no longer constrained or otherwise held in an elongated configuration by) the delivery lumen 315, the memory material section 2014 shifts to its deflected configuration, as illustrated in FIG. 3A, preferably without application of external forces thereto (e.g., independently, as a result of natural bending forces of the memory material section 2014). If desired, one or more compliant material sections 2016 may be provided to facilitate bending or flexing of the distal end 2011 of the deflectable flexible elongate member 2010.

In some instance, a visualization/imaging system may be delivered to an implant site in a position biased with respect to the implant site and/or the device to be implanted. For instance, even a visualization/imaging system delivered with the implantable device (e.g., within an area circumscribed by the implantable device) may be biased or skewed relative to the implantable device and thus closer to one side of the implantable device than another side. As the distance between the imaging element of a visualization/imaging system and the device or site or object to be imaged may affect the quality of the image, it is desirable to be able to adjust such distance as desired or needed. To adjust the position of the imaging element and, generally, the view of the visualization/imaging system 2000 relative to the implantable device 100 and/or the heart valve annulus VA, the deflectable flexible elongate member 2010 may be moved, such as rotated (such as generally described above) or shifted laterally. As illustrated in FIG. 3B, if the deflectable flexible elongate member 2010 is shifted laterally, the angular position and/or orientation of the imaging element 2020 on the distal end 2011 of the deflectable flexible elongate member 2010 may be adjusted relative to the longitudinal axis LA of the flexible elongate member 2010 as well as relative to the frame and/or the implant site at which the visualization/imaging system 2000 is positioned.

In accordance with various principles of the present disclosure, as noted above, a visualization/imaging system may include one or more imaging elements for visualizing or imaging an implantable device and/or implant site. A deflectable visualization/imaging system 2000 such as illustrated in FIGS. 3A and 3B may include two or more imaging elements 2020, 2020′ circumferentially spaced apart from one another. For instance, a first imaging element 2020, illustrated in solid lines, may be positioned on a first proximally-facing (upward-facing) side of the distal end 2011 of the deflectable flexible elongate member 2010, and an optional second imaging element 2020′, illustrated in broken lines, may be provided on the distally-facing (lower-facing) side of the deflected distal end 2011 of the deflectable flexible elongate member 2010. Additional or alternative positions of imaging elements 2020, 2020′ to provide different view angles or fields are within the scope of the present disclosure.

Additionally or alternatively, a visualization/imaging system formed in accordance with various principles of the present disclosure may include two or more imaging elements longitudinally spaced apart from one another. For instance, in the example of an embodiment illustrated in FIG. 4, a visualization/imaging system 3000 may have a deflectable flexible elongate member 3010 (e.g., with a memory material section 3014 causing the distal end 3011 to deflect independently once deployed outside the delivery catheter 310) with a first transducer 3020a on the distal end 3011 thereof, and a second transducer 3020b proximal to the first imaging element 3020 and optionally spaced apart from the first imaging element 3020b. Both imaging elements 3020a, 3020b may be considered to be positioned on the distal end 3011 of the flexible elongate member 3010, with the first imaging element 3020a being closer to the free end (the distalmost end not connected to another element) of the flexible elongate member 3010. In the example illustrated in FIG. 4, the first and second imaging elements 3020a, 3020b are positioned on different sides (in a longitudinal direction) with respect to the bend 3012, e.g., with the first imaging element 3020a distal to the bend 3012 and the second imaging element 3020b proximal to the bend 3012. As such, the imaging elements 3020a, 3020b are not only at different positions, but also are at different orientations (e.g., angles) with respect to each other as well as with respect to the implantable device 100 and/or the heart valve annulus VA, and thus provide various viewing areas (distal or proximal sides of the implantable device 100, and/or atrial or ventricular sides of the valve annulus VA) and angles. It will be appreciated that additional imaging elements may be provided longitudinally or circumferentially with respect to imaging elements 3020a, 3020b, such as spaced apart longitudinally or circumferentially. For instance, an additional imaging element 3020c (shown in broken lines) may be provided on a side of the distal end 3011 of the deflectable flexible elongate member 3010 opposite the first imaging element 3020a, such as to face downwardly, similar to in the example of an embodiment illustrated in FIG. 3B.

As noted above, a visualization/imaging system formed in accordance with various principles of the present disclosure may be steerable in addition to or instead of being deflectable on its own accord (e.g., formed from a shape memory material to be self-deflecting without external forces). Any of a variety of steering mechanisms such as described in further detail below may be used to steer a flexible elongate member of a visualization/imaging system formed in accordance with various principles of the present disclosure. It will be appreciated that, as described above, one or more imaging elements may be provided along the distal end of the flexible elongate member, such as longitudinally or circumferentially with respect to one another, and optionally spaced apart from one another.

An example of an embodiment of a steerable visualization/imaging system 4000 is illustrated in FIG. 5 without details of the steering mechanism being illustrated. The illustrated visualization/imaging system 4000 has a steerable flexible elongate member 4010 with a distal end 4011. A first imaging element 4020a is positioned along a first side of the distal end 4011 of the flexible elongate member 4010, and a second imaging element 4020b is spaced apart from the first imaging element 4020b, such as along a side of the distal end 4011 of the flexible elongate member 4010 opposite the first side. Although the flexible elongate member 4010 may be rotated to view different areas or regions around the visualization/imaging system 4000, bending of the distal end 4011 in a first direction D₁ towards the first imaging element 4020a (to move the first imaging element 4020a proximally or to face upwardly) or in a second direction D₂ towards the second imaging element 4020b (to move the second imaging element 4020b proximally or to face upwardly) will vary the viewing area of the visualization/imaging system 4000 while reducing the degree to which the flexible elongate member 4010 is rotated (which may be complicated if the flexible elongate member 4010 is extended along a tortuous pathway through a body), and will increase the area of viewing by varying the angle of the imaging elements 4020a, 4020b relative to the implantable device 100 and the heart valve annulus VA. Optionally, the distal end 4011 of the flexible elongate member 4010 may include one or more memory material sections 4014 and, optionally, one or more compliant material sections 4016 which facilitate the flexing of the distal end 4011 of the flexible elongate member 4010 independently of and/or in conjunction with a steering mechanism.

In accordance with various principles of the present disclosure, more than one imaging element may be provided on a distal end of a visualization/imaging system by being provided on more than one distal end of the visualization/imaging system. More particularly, the visualization/imaging system may have more than one distal end, each distal end carrying one or more imaging elements. The distal ends may be movable independently of or together with one another to vary the viewable area of the visualization/imaging system (e.g., the imaging elements thereof) relative to the implantable device 100 and/or the heart valve annulus VA. One or more of the distal ends may be deflectable and/or steerable. Examples of visualization/imaging systems with more than one distal end are illustrated in FIGS. 6 and 7.

In the example of an embodiment illustrated in FIG. 6, a visualization/imaging system 5000 has a flexible elongate member 5010 with coextensive first and second flexible elongate members 5010a, 5010b. Such configuration may be considered a dual monorail or mother-daughter configuration. More particularly, the flexible elongate member 5010 includes a first flexible elongate member 5010a having a lumen 5015 defined therethrough through which a second flexible elongate member 5010b extends. As such, the second flexible elongate member 5010b is at least translatable with respect to the first flexible elongate member 5010a. Optionally, one or both of the flexible elongate members 5010a, 5010b are rotatable, such as with respect to each other, as well. As illustrated, the first flexible elongate member 5010a may be extended through a heart valve annulus VA in which an implantable device 100 is being implanted, and then flexed, such as to visualize the ventricular side of the heart valve annulus VA. The second flexible elongate member 5010b may be rotatable to vary the view and visualization area, and/or may be r deflectable or steerable into a desired angular position with respect to the first flexible elongate member 5010a to achieve a desired viewing field. The second flexible elongate member 5010b may remain on the atrial side of the heart valve annulus VA to visualize the implantable device 100 and/or may be positioned on the ventricular side of the heart valve annulus VA with the first flexible elongate member 5010a. Either or both of the first and second flexible elongate members 5010a, 5010b may be adjusted (translated, rotated, flexed, etc.) to adjust the field of view of the one or more imaging elements 5020 carried by such flexible elongate member 5010a, 5010b.

In the example of an embodiment illustrated in FIG. 7, a visualization/imaging system 6000 has a flexible elongate member 6010 with a bifurcated distal end 6011 forming first and second distal ends 6011a, 6011b of the flexible elongate member 6010. One or both of the flexible distal ends 6011a, 6011b are deflectable (such as by memory material sections 6014) and/or steerable, and preferably may be moved independently of each other. As illustrated, the first distal end 6011a may be extended through a heart valve annulus VA in which an implantable device 100 is being implanted and then steered (such as with a steering mechanism) and/or deflected, such as to visualize the ventricular side of the heart valve annulus VA. The imaging element 6020 on the first distal end 6011a may be on a proximally facing side of the first distal end 6011a once the first distal end 6011a has been steered or deflected, such as illustrated. The second distal end 6011b may be deflectable (e.g., without external force, such as by one or more memory material sections 6014) and or steerable to vary the view and visualization area. The second distal end 6011b may remain on the atrial side of the heart valve annulus VA to visualize the implantable device 100 and/or may be positioned on the ventricular side of the heart valve annulus VA with the first flexible elongate member 5010a. The imaging element 6020 on the second distal end 6011b may be on a distally-facing side of the second distal end 6011b once the second distal end 6011b has been deflected, such as illustrated.

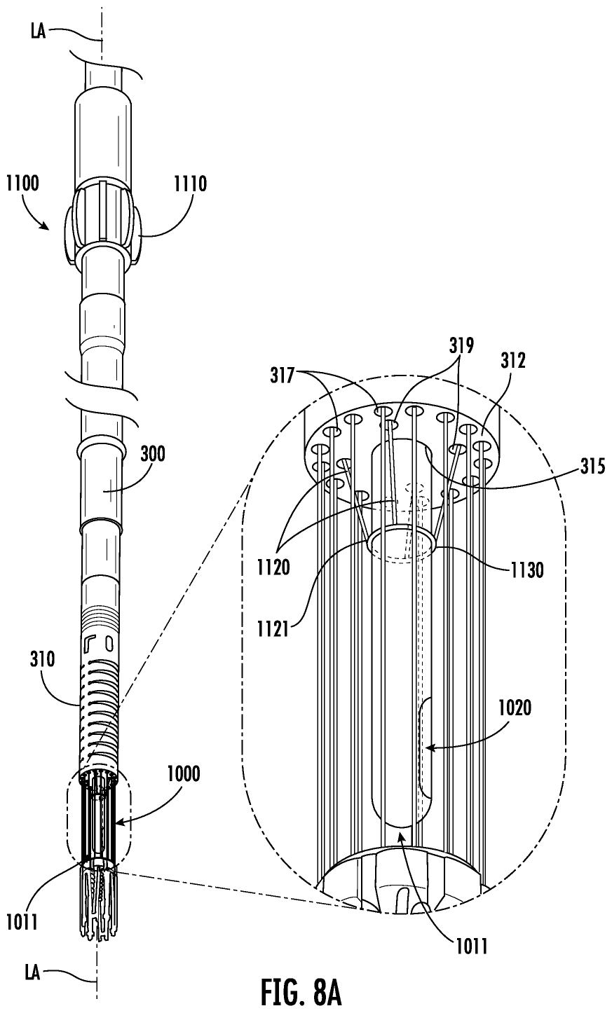

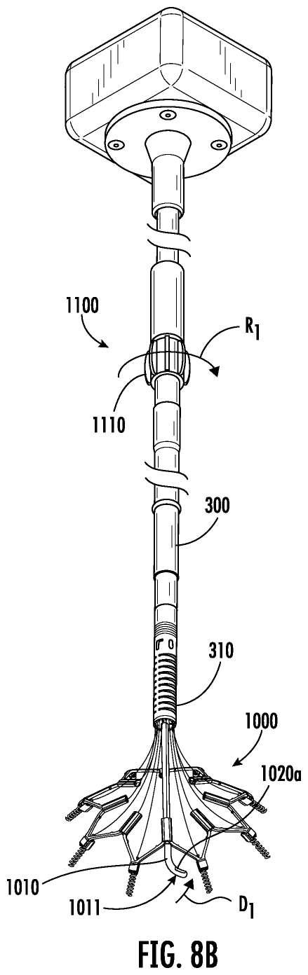

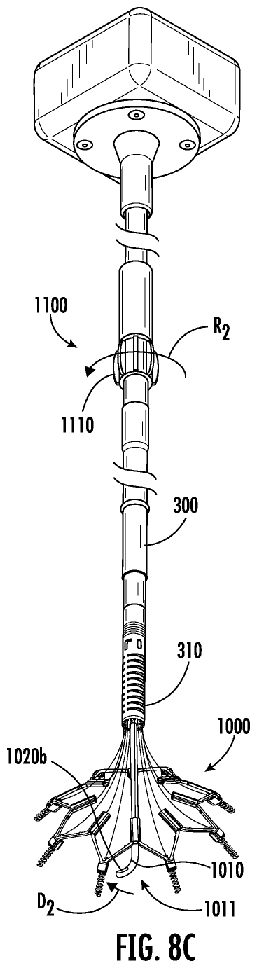

The above-described steering of a visualization/imaging system formed in accordance with various principles of the present disclosure may be achieved by any of a variety of known or heretofore known steering mechanisms The steering mechanism preferably allows the visualization/imaging system to be maneuvered as desired from a convenient location proximal to the device, and preferably outside the patient. The steering mechanism may be controlled manually or automatically (such as computer-controlled). Embodiments are not limited in this context. Various examples of steering mechanisms which may be used to steer a flexible elongate member (and particularly the distal end thereof) of a visualization/imaging system formed in accordance with various principles of the present disclosure will now be described with reference to FIGS. 8A, 8B, 8C, and 9-12.

An example of an embodiment of a visualization/imaging system 1000 with a steering mechanism 1100 having a rotatable controller 1110 is illustrated in FIGS. 8A, 8B, 8C. The rotatable controller 1110 is rotatable about a rotational axis substantially parallel to the longitudinal axis LA of the steerable flexible elongate member 1010 of the visualization/imaging system 1000. As may be appreciated, the steerable flexible elongate member 1010 may be positioned in a substantially straight configuration, to be understood as substantially aligned with the longitudinal axis LA of the delivery catheter 310 and extending through delivery lumen 315 defined therethrough. The visualization/imaging system 1000 and its steerable flexible elongate member 1010 extend proximally, from the one or more imaging elements 1020 at the distal end 1011 of the steerable flexible elongate member 1010, and through the delivery catheter 310 and the access catheter 300, to the controller 1110. The controller 1110 preferably is outside the patient's body while the distal end 1011 of the steerable flexible elongate member 1010 is within the patient, such as at the implant site.

In the examples of embodiments illustrated in FIGS. 8A, 8B, 8C, steering wires 1120 extend from the distal end 1011 of the steerable flexible elongate member 1010 to the controller 1030. The distal ends 1121 of the steering wires 1120 are coupled to the distal end 1011 of the steerable flexible elongate member 1010, such as via a pull ring 1130. For instance, the steering wires 1120 may be bonded, fused, welded, adhered, etc., to the pull ring 1130, and the pull ring 1130, in turn, may be bonded, fused, welded, adhered, etc., to the steerable flexible elongate member 1010. The flexible elongate member 1010 may have a reduced outer diameter proximal to the pull ring 1130, as illustrated, or may have a substantially constant outer diameter from its distal end 1011 to its proximal end (outside the body as may be appreciated by one of ordinary skill in the art, and not illustrated in the drawings). It will be appreciated that other manners of operatively coupling a controller 1100 with the flexible elongate member 1010 are within the scope and spirit of the present disclosure, such details not being critical to an understanding of the present disclosure. In some embodiments, the delivery catheter 310 is formed with a multi-lumen extrusion 312 through which a plurality of steering control lumens 319 are axially defined. The steering wires 1120 may extend through the steering control lumens 319. In some embodiments of a delivery catheter 310 formed with a multi-lumen extrusion 312, the multi-lumen extrusion 312 defines device manipulation lumens 317 (through which driver shafts 140 extend to adjust the implantable device 100, such as illustrated in FIG. 1 and FIG. 2) as well as steering control lumens 319. In such embodiment, the steering control lumens 319 may be positioned radially inwardly of the device manipulation lumens 317 if the visualization/imaging system 1000 is to be positioned within the circumscribed area A within the implantable device 100. In other embodiments, steering wires 1120 may extend through a separate steering sheath (not shown) extended over the flexible elongate member 1010.

Rotation of the controller 1110 in a first rotational direction R₁, as illustrated in FIG. 8B, actuates (e.g., pulls or pushes) one or more of the steering wires 1120 to cause the distal end 1011 of the steerable flexible elongate member 1010 to move in a first direction D₁, such as to move a first imaging element 1020a to face proximally. Rotation of the controller 1110 in a second rotational direction R₂. as illustrated in FIG. 8B, actuates (e.g., pulls or pushes) one or more of the steering wires 1120 to cause the distal end 1011 of the steerable flexible elongate member 1010 to move in a second direction D₂, such as to move a second imaging element 1020b (on a side of the steerable flexible elongate member 1010 opposite the side on which the first transducer 1020a is located) to face proximally. It will be appreciated that other movements are within the scope and spirit of the present disclosure. It will further be appreciated that if more than one imaging element 1020 is provided about the perimeter of the flexible elongate member 1010, then the distal end 1011 of the flexible elongate member 1010 may be deflected in only one direction to obtain images in an upward as well as a downward direction.

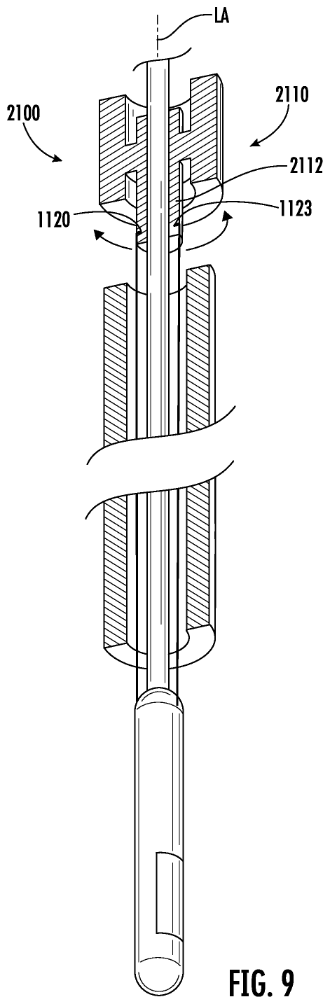

A rotatable controller 1110, as in the examples of embodiments illustrated in FIGS. 8A, 8B, and 8C, may effectuate movement of a steerable flexible elongate member 1010 in any manner known or heretofore known. For example, in the example of an embodiment of a steering mechanism 2100 illustrated in FIG. 9, a rotatable controller 2110 may include a spool 2112 rotatable about a rotational axis substantially parallel to the longitudinal axis LA of the steerable flexible elongate member 1010. The proximal ends 1121 of the steering wires 1120 may be coupled to the outer surface of the spool 2112 such that rotation of the spool 2112 pulls or pushes the steering wires 2120 to cause move of the distal end 1011 of the steerable flexible elongate member 1010 in a manner as may be readily appreciated by one of ordinary skill in the art with reference to FIG. 9.

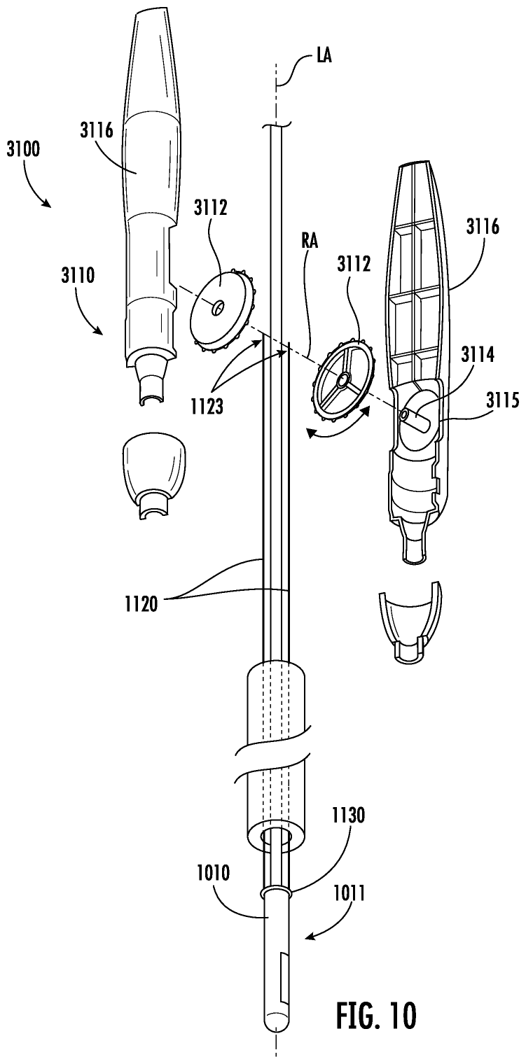

It will be appreciated that steering mechanisms formed in accordance with various principles of the present disclosure may include any of a variety of other types of controllers. For example, instead of having a controller which rotates about a rotational axis substantially parallel to the longitudinal axis LA of the steerable flexible elongate member 1010, a steering mechanism controller may be rotatable about a rotational axis RA transverse to (e.g., perpendicular to) the longitudinal axis LA of the steerable flexible elongate member 1010. For instance, in the example of an embodiment of a steering mechanism 3100 illustrated in FIG. 10, a rotatable controller 3110 may include a spool or wheel 3112 to which proximal ends 1023 of the steering wires 1020 are coupled. The wheel 3112 is mounted on an axle 3114 extending generally transverse to (e.g., perpendicular to) the longitudinal axis LA of the steerable flexible elongate member 1010. The proximal ends 1123 of the steering wires 1120 are coupled to the wheel 3112 spaced apart from the rotational center of the wheel 3112 (and spaced apart from the rotational axis RA). The wheel 3112 may be mounted on an axle 3114 within a housing 3116 of the controller 3110. Portions of the wheel 3112 may be accessible through windows 3115 of the housing 3114 to rotate the wheel 3112 in a clockwise or counterclockwise direction about the axle 3114. Rotation of the wheel 3112 about the rotational axis RA moves (e.g, pulls/pushes) the steering wires 1120 to flex the distal end 1011 of the steerable flexible elongate member 1010 (such as via the pull ring 1130) in a manner as may be readily appreciated by one of ordinary skill in the art with reference to FIG. 10.

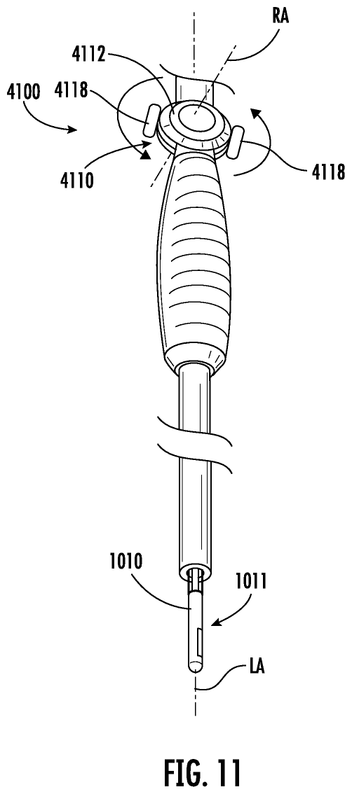

Another example of an embodiment of a steering mechanism 4100 with a rotatable controller 4110 actuated by rotating about a rotational axis RA transverse to the longitudinal axis LA of the steerable flexible elongate member 1010 is illustrated in FIG. 11. Instead of an internally positioned wheel 3112 as in the example of an embodiment illustrated in FIG. 10, the controller 4110 of the example of an embodiment illustrated in FIG. 11 has an externally positioned wheel 4112 with a control surface 4118 on either side thereof. The control surfaces 4118 are configured to facilitate contact and rotational movement of the wheel 4112 about the rotational axis RA to shift the steering wires 1120 (coupled internally within the wheel 4112 generally spaced apart from the rotational axis RA) to cause the distal end 1011 of the steerable flexible elongate member 1010 to move as may be appreciated by one of ordinary skill in the art with reference to FIG. 11.

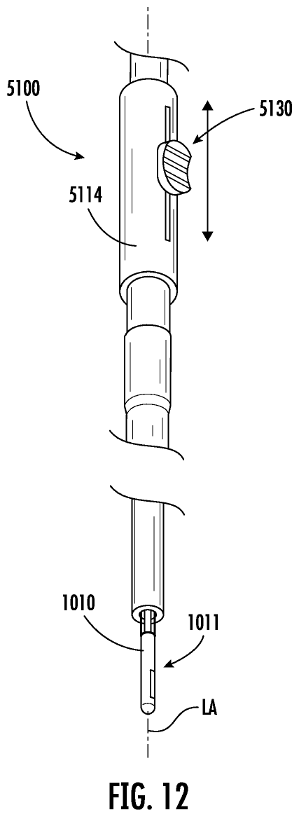

As an alternative to a rotatable controller, as in the examples of embodiments illustrated in FIGS. 8A, 8B, 8C, and 9-11, a steering mechanism formed in accordance with various principles of the present disclosure to steer the distal end 1011 of a steerable flexible elongate member 1010 of a visualization/imaging system 1000 formed in accordance with various principles of the present disclosure may include a slidable controller. An example of an embodiment of a slidable controller 5030 is illustrated in FIG. 12. The slidable controller 5110 is slidably mounted on a housing 5114 to slide along the longitudinal axis LA of the steerable flexible elongate member 1010. The steering wires 1120 coupled to distal end 1011 of the steerable flexible elongate member 1010 may be coupled to the controller 5110 internally within the housing 5114 in any desired manner such that movement of the controller 5110 causes movement of the steering wires 1120 to steer the distal end 1011 of the steerable flexible elongate member 1010 in a manner as may be appreciated by one of ordinary skill in the art with reference to FIG. 12.

As may be appreciated, the ability of a visualization/imaging system to be moved with respect to an implantable device in accordance with any one or more of the various principles of the present disclosure described herein improves and/or increases and/or enhances the visualization/imaging field of the visualization/imaging system. The implantable device may be any type of implantable device such as described herein or otherwise. Improved or increased or enhanced visualization/imaging may be used to image the implant site from various positions, verify the positioning of the implantable device relative to the implant site, determine the condition of the tissue at the implant site, determine the depth of insertion of the implanted components of the implantable device, etc. For instance, in connection with annuloplasty devices, the annulus, ventricle, chordae may be imaged from various positions (e.g., the upper or atrial side, and/or the underside or ventricular side) depending upon anatomy anomalies/disease; the position of one or more anchors of the implantable device with respect to the heart valve annulus VA and/or the leaflets may be verified; the condition of the tissue of the heart valve VA may be determined; the depth of insertion of an anchor may be determined, etc. As such, proper delivery, positioning, placement, anchoring, etc. of the implantable device 100, such as to reshape the valve annulus or replace the entire valve, are achievable in manners not as readily achievable previously. Examples of embodiments of visualization/imaging methods are described in detail herein with reference to the figures.

Generally, in accordance with one aspect of the present disclosure the method includes advancing a deployment catheter, such as the access and/or delivery catheters described herein, to a deployment site in a heart, with the implantable device. The implantable device may have at least one tissue anchor, such as the anchors described herein. An imaging element, such as the ICE or ultrasound catheters described herein, is positioned in the heart adjacent the implantable device. A relationship is visualized between the tissue anchor and an anatomical landmark in the heart, and the implantable device is attached by driving the tissue anchor into tissue in the heart.

In some embodiments, the method generally includes advancing a distal end of a catheter, such as the access and/or delivery catheters described herein, proximate an implant site, such as the heart valve annulus in the heart. The implantable device (e.g., heart valve replacements having valve leaflets or mitral valve leaflet repair devices) is advanced through the distal end of the catheter proximate the implant site (e.g., the heart valve annulus). A distal end of an ultrasound catheter, such as the ICE or other ultrasound catheters described herein, is advanced proximate the implantable device/implant site. The distal end of the ultrasound catheter includes one or more imaging elements, such as ultrasonic transducers. An image of the implantable device and/or the implant site is transmitted and/or captured with the one or more imaging elements, and the implantable device is anchored to the implant site. The images may be used to verify the position of, and/or re-position, the anchors before driving the anchors into the tissue at the implant site. The images may additionally or alternatively be used to verify the configuration of the implantable device, such as once anchored to the implant site, such as to modify or reconfigure the shape of the heart valve annulus.

The implantable device may be inserted into the implant site using a delivery/deployment system, such as (without limitation) any of the delivery/deployment systems described herein. The implantable device and the delivery/deployment system may be inserted via access to the vasculature of the leg, in particular the femoral vein or the iliac vein. A visualization/imaging system, such as (without limitation) any of the visualization/imaging systems described herein may be inserted with the implantable device and the visualization/imaging system. The implantable device, delivery/deployment system, and/or visualization/imaging system may then be advanced across the septum separating the upper chambers of the heart.

In the examples of embodiments described herein, the imaging elements are advanced to a position above the heart valve annulus, for example, the mitral valve annulus. The implantable device may be deployed before the visualization/imaging system, or the visualization/imaging system may be delivered with the implantable device through a common access sheath. The visualization/imaging system may be positioned by being advancing through a lumen in a delivery catheter, such as of the delivery/deployment system of the implantable device. In some embodiments, the visualization/imaging system may be positioned by being advanced transvascularly along a different path than that followed by the delivery/deployment system. It will be appreciated that a guidewire may be used to guide any or all devices or systems, such as by being extended through the annulus and into the left ventricle. Although reference is generally made herein to the left ventricle and the mitral valve, it will be appreciated that the devices, systems, and methods described herein may be used to treat the tricuspid valve. For example, the delivery/deployment system and implantable device may be inserted for access through the jugular vein and advanced down the superior vena cava and into the right atrium proximate and above the tricuspid valve annulus.

In some embodiments, the visualization/imaging system is positioned within an area circumscribed by the implantable device, or otherwise positioned adjacent the implantable device to facilitate viewing and imaging of the implantable device.

In some embodiments, the visualization/imaging system includes a flexible elongate member having a longitudinal axis, and an imaging element positioned on a portion of the flexible elongate member, such as the distal end of the flexible elongate member. For the sake of convenience, and without intent to limit, reference is made to the distal end of the flexible elongate member as the portion on which the imaging element is positioned. The imaging element is moved with respect to the longitudinal axis of the flexible elongate member to adjust the position and/or orientation of the imaging element with respect to the implantable device and/or the implant site. The distal end of the flexible elongate member may deflect on its own (without external forces moving the distal end, such as a result of internal forces of a memory material section of the distal end), or may be steered or otherwise moved by an external force. In some embodiments, the method comprises deflecting or steering the imaging element in the direction of one or more anchors of the implantable device. In some embodiments, a first image of a first anchor is captured, the imaging element is then repositioned, and then a second image of a second anchor is captured, and such process is continued until the anchors are positioned and/or implanted as desired. In some embodiments, the repositioning comprises rotating the flexible elongate member and the imaging element about an axis (e.g., the longitudinal axis of the flexible elongate member) using a rotational drive mechanism and/or by manually rotating the flexible elongate member about the axis. In some embodiments, the flexible elongate member carries the imaging element (such as a transducer), and the imaging element is rotated, deflected, etc. as described. In some embodiments, a proximal engagement structure on the visualization/imaging system is locked to a complementary engagement structure on the delivery/deployment system of the implantable device. In some embodiments, the angular orientation of the imaging element is adjusted, such as relative to the longitudinal axis of the flexible elongate member, to adjust the orientation of the imaging element relative to the implantable device and/or the implant site. For instance, it may be advantageous for the imaging element to be oriented generally parallel to an anchor or other component of the implantable device. It will be appreciated that one or more imaging elements may be provided, the positions of which may be adjusted with respect to one another.

At least one radial image is taken with the visualization/imaging system, such as with a phased array of transducers. It will be appreciated that reference to a radial image may be understood to include, without limitation, a cross-sectional image, such as encompassing a cross-sectional image of the implantable device positioned with respect to or in the implant site. A series of radial images or multiple images of the implantable device and/or the implant site may be taken. Additionally or alternatively, the imaging element is rotated to a plurality of rotational positions, and a series of images or multiple images of the implantable device and/or the implant site are captured/generated and transmitted, The images are used to properly implant the implantable device, such as to properly position anchors of the implantable device for insertion into mitral valve annulus tissue. The implantable device may be confirmed, with reference to the images transmitted by the visualization/imaging system, to be in the proper position, orientation, etc., and implanted into the cardiac tissue, for example by rotating the one or more (e.g., at least two, at least six, or at least eight) anchors thereof into tissue. The images may be used to confirm that the implantable device has been properly positioned and/or implanted. For instance, in the case of an annuloplasty device, the images may be used to confirm that all anchors have been appropriately placed and anchored in the mitral valve annulus tissue above the mitral valve leaflets. If one or more anchors are not positioned or anchored properly, they can be manipulated, for example rotationally retracted, repositioned, and re-anchored, prior to removal of the driver shafts. In addition, images can be taken prior to anchoring to confirm location of portions of the implantable device, such as the distal portion of the implantable device to be secured to the implant site. The various images may be used to visualize various anatomical features of the heart, such as the heart valve annulus, the heart valve, valve leaflets, the mitral valve, the tricuspid valve, and/or other features. In addition or alternatively, the various images may be used to visualize various features of the delivery/deployment system, the implantable device, and/or components of the implantable device (such as the anchors), etc. The implantable device may be released from the delivery/deployment system following capturing the images and confirmation of proper and successful implantation at the implant site.

Although embodiments of the present disclosure may be described with specific reference to positioning an implantable device for use with mitral valves, it is appreciated that various other implantable devices may similarly benefit from the device, systems, and methods disclosed herein.

Various modifications to the implementations described in this disclosure will be readily apparent to those skilled in the art, and the generic principles defined herein can be applied to other implementations without departing from the spirit or scope of this disclosure. Thus, the disclosure is not intended to be limited to the implementations shown herein, but is to be accorded the widest scope consistent with the claims, the principles and the novel features disclosed herein. The word “example” is used exclusively herein to mean “serving as an example, instance, or illustration.” Any implementation described herein as “example” is not necessarily to be construed as preferred or advantageous over other implementations, unless otherwise stated.

Certain features that are described in this specification in the context of separate implementations also can be implemented in combination in a single implementation. Conversely, various features that are described in the context of a single implementation also can be implemented in multiple implementations separately or in any suitable sub-combination. Moreover, although features can be described above as acting in certain combinations and even initially claimed as such, one or more features from a claimed combination can in some cases be excised from the combination, and the claimed combination can be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. Additionally, other implementations are within the scope of the following claims. In some cases, the actions recited in the claims can be performed in a different order and still achieve desirable results.

In view of the above, it should be understood that the various embodiments illustrated in the figures have several separate and independent features, which each, at least alone, has unique benefits which are desirable for, yet not critical to, the presently disclosed devices, systems, and methods. Therefore, the various separate features described herein need not all be present in order to achieve at least some of the desired characteristics and/or benefits described herein. Only one of the various features described herein may be present in an embodiment in accordance with various principles of the present disclosure. Alternatively, one or more of the features described with reference to one embodiment can be combined with one or more of the features of any of the other embodiments provided herein. That is, any of the features described herein can be mixed and matched to create hybrid designs, and such hybrid designs are within the scope of the present disclosure. Moreover, throughout the present disclosure, reference numbers are used to indicate a generic element or feature of the disclosed embodiment. The same reference number may be used to indicate elements or features that are not identical in form, shape, structure, etc., yet which provide similar functions or benefits. Additional reference characters (such as letters, as opposed to numbers) may be used to differentiate similar elements or features from one another.

All apparatuses and methods discussed herein are examples of apparatuses and/or methods implemented in accordance with one or more principles of this disclosure. These examples are not the only way to implement these principles but are merely examples, not intended as limiting the broader aspects of the present disclosure. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure.

The foregoing discussion has broad application and has been presented for purposes of illustration and description and is not intended to limit the disclosure to the form or forms disclosed herein. It will be understood that various additions, modifications, and substitutions may be made to embodiments disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof. For example, various features of the disclosure are grouped together in one or more aspects, embodiments, or configurations for the purpose of streamlining the disclosure. However, it should be understood that various features of the certain aspects, embodiments, or configurations of the disclosure may be combined in alternate aspects, embodiments, or configurations. While the disclosure is presented in terms of embodiments, it should be appreciated that the various separate features of the present subject matter need not all be present in order to achieve at least some of the desired characteristics and/or benefits of the present subject matter or such individual features. One skilled in the art will appreciate that the disclosure may be used with many modifications or modifications of structure, arrangement, proportions, materials, components, and otherwise, used in the practice of the disclosure, which are particularly adapted to specific environments and operative requirements without departing from the principles or spirit or scope of the present disclosure. For example, elements shown as integrally formed may be constructed of multiple parts or elements shown as multiple parts may be integrally formed, the operation of elements may be reversed or otherwise varied, the size or dimensions of the elements may be varied. Similarly, while operations or actions or procedures are described in a particular order, this should not be understood as requiring such particular order, or that all operations or actions or procedures are to be performed, to achieve desirable results. Additionally, other implementations are within the scope of the following claims. In some cases, the actions recited in the claims can be performed in a different order and still achieve desirable results. The presently disclosed embodiments are therefore to be considered in all respects as illustrative and not restrictive, the scope of the claimed subject matter being indicated by the appended claims, and not limited to the foregoing description or particular embodiments or arrangements described or illustrated herein. In view of the foregoing, individual features of any embodiment may be used and can be claimed separately or in combination with features of that embodiment or any other embodiment, the scope of the subject matter being indicated by the appended claims, and not limited to the foregoing description.

In the foregoing description and the following claims, the following will be appreciated. The phrases “at least one”, “one or more”, and “and/or”, as used herein, are open-ended expressions that are both conjunctive and disjunctive in operation. The terms “a”, “an”, “the”, “first”, “second”, etc., do not preclude a plurality. For example, the term “a” or “an” entity, as used herein, refers to one or more of that entity. As such, the terms “a” (or “an”), “one or more” and “at least one” can be used interchangeably herein. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader's understanding of the present disclosure, and/or serve to distinguish regions of the associated elements from one another, and do not limit the associated element, particularly as to the position, orientation, or use of this disclosure. Connection references (e.g., attached, coupled, connected, and joined) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another.

The following claims are hereby incorporated into this Detailed Description by this reference, with each claim standing on its own as a separate embodiment of the present disclosure. In the claims, the term “comprises/comprising” does not exclude the presence of other elements or steps. Additionally, although individual features may be included in different claims, these may possibly advantageously be combined, and the inclusion in different claims does not imply that a combination of features is not feasible and/or advantageous. In addition, singular references do not exclude a plurality. Reference signs in the claims are provided merely as a clarifying example and shall not be construed as limiting the scope of the claims in any way.

What is claimed is:

1. A method of deploying an implantable device, the method comprising:
delivering an implantable device and a visualization/imaging system through a common lumen defined through an access sheath to an implant site; and
causing a distal end of a flexible elongate member of the visualization/imaging system to move with respect to the longitudinal axis of the flexible elongate member to move an imaging element on the distal end of the visualization/imaging system relative to the implantable device and/or the implant site.

2. The method of claim 1, further comprising moving the imaging element to alter an angle of the imaging element relative to the implantable device and/or the implant site to affect visualization thereof.

3. The method of claim 1, further comprising:
visualizing and/or imaging the implantable device with the visualization/imaging system; and
anchoring a portion of the implantable device to the implant site based on visualizations and/or images from the visualization/imaging system.

4. The method of claim 1, further comprising:
visualizing and/or imaging the implantable device with the visualization/imaging system; and
adjusting a configuration of the implantable device based on visualizations and/or images from the visualization/imaging system.

5. The method of claim 1, further comprising delivering the visualization/imaging system into an area circumscribed by the implantable device.

6. The method of claim 5, further comprising delivering the implantable device with the use of a delivery/deployment system having a delivery lumen, the visualization/imaging system being delivered through the delivery lumen of the delivery/deployment system.

7. The method of claim 1, wherein the flexible elongate member includes at least one shape memory section such that bending of the flexible portion of the flexible elongate member is achieved without application of external force to the flexible elongate member.

8. The method of claim 1, further comprising using a steering mechanism to bend the flexible portion of the flexible elongate member.

9. The method of claim 1, further comprising moving the imaging element to view a distal side of the implantable device and/or the implant site.

10. A method of deploying an implantable device, the method comprising:
delivering an implantable device to an implant site;
delivering a visualization/imaging system adjacent the implantable device, the visualization/imaging system including a flexible portion on which at least one imaging element is positioned;
visualizing and/or imaging the implantable device with the visualization/imaging system; and
bending the flexible portion of the flexible elongate member to alter the position of the at least one imaging element relative to the implantable device and/or the implant site to adjust the visualizing and/or imaging performed by the visualization/imaging system.

11. The method of claim 10, further comprising anchoring a portion of the implantable device to the implant site based on visualizations and/or images from the visualization/imaging system.

12. The method of claim 10, further comprising adjusting a configuration of the implantable device based on visualizations and/or images from the visualization/imaging system.

13. The method of claim 10, further comprising delivering the visualization/imaging system into an area circumscribed by the implantable device.

14. The method of claim 10, wherein the flexible elongate member includes at least one shape memory section such that bending of the flexible portion of the flexible elongate member is achieved without application of external force to the flexible elongate member.

15. The method of claim 10, further comprising using a steering mechanism to bend the flexible portion of the flexible elongate member.

16. The method of claim 10, wherein bending the flexible portion of the flexible elongate member alters the angle of the at least one imaging element relative to the implantable device and/or the implant site.

17. The method of claim 10, further comprising moving the imaging element to view a distal side of the implantable device and/or the implant site.

18. A system for implanting an implantable device at an implant site, the system comprising:
an implantable device circumscribing an area; and
a visualization/imaging system with at least one imaging element;
wherein:
the at least one imaging element is positionable within the circumscribed area within the implantable device and movable with respect to the implantable device to alter the angle of the imaging element relative to a portion of the implantable device to enhance visualization or imaging of the portion of the implantable device.

19. The system of claim 18, wherein:
the visualization/imaging system includes a flexible elongate member extending along longitudinal axis and having a flexible portion;
the at least one imaging element is positioned adjacent the flexible portion of the flexible elongate member; and
bending of flexible portion of the flexible elongate member alters the angle of the imaging element relative to the longitudinal axis of the flexible elongate member and the implantable device.

20. The system of claim 18, wherein:
the visualization/imaging system includes a flexible elongate member extending along longitudinal axis; and
the at least one imaging element comprises a first imaging element on a distal end of the flexible elongate member and a second imaging element positioned adjacent and proximal to the first imaging element;
wherein the flexible elongate member is bendable between the first imaging element and the second imaging element to alter the angle between the first and second imaging elements to visualize and/or image different portions of the implantable device.
